(19) United States
(12) Patent Application Publication
(10) Pub. No.: US 2013/0115295 A1
(43) Pub. Date: May 9, 2013

(54) Rare Earth-Doped Up-Conversion Nanoparticles for Therapeutic and Diagnostic Applications

(71) Applicants: Qiang Wang, Highland Park, NJ (US); Patrick Y. Lu, Rockville, MD (US); Harry Hongjun Yang, Derwood, MD (US)

(72) Inventors: Qiang Wang, Highland Park, NJ (US); Patrick Y. Lu, Rockville, MD (US); Harry Hongjun Yang, Derwood, MD (US)

(21) Appl. No.: 13/511,342

(22) PCT Filed: Nov. 22, 2010
(86) PCT No.: PCT/US10/57679

§ 371 Date: Jan. 11, 2013

Related U.S. Application Data
(60) Provisional application No. 61/263,392, filed on Nov. 22, 2009.

(51) Int. Cl. A61K 41/00; G01N 33/58

(52) U.S. Cl. CPC A61K 41/0042; G01N 33/582
USPC 424/491; 424/490; 514/44 A; 436/94

(57) ABSTRACT

This invention provides a composition matter comprising rare earth-doped up-conversion nanoparticles (UCNPs) encapsulated with a silica shell. In one embodiment, a photosensitizer is incorporated into the silica shell. In another embodiment, the composition further comprises a targeting molecule. In still another embodiment, a small interfering RNA (siRNA) molecule is also attached to the silica shell with the targeting molecule. The invention further provides methods for synthesizing such compositions and for using them in therapeutic and diagnostic applications. These applications use infrared or near infrared activation to excite the UCNPs.

### CROSS-REFERENCE TO RELATED PATENT APPLICATIONS

This application claims the benefit of and priority to U.S. Provisional Patent Application No. 61/263,392, filed Nov. 22, 2009, which is incorporated herein by reference in its entirety.

### FIELD OF THE INVENTION

The present invention relates to a composition of matter comprising rare earth-doped up-conversion nanoparticles (UCNPs) encapsulated with a silicon shell, which enhances chemodrug, small interfering RNA (siRNA) inhibitor, or peptide release, and to methods for synthesizing such particles and using them for treatment of various human diseases. An example is the treatment of cancer, using an siRNA inhibitor with the assistance of the UCNPs. An infrared light is used to excite up-conversion nanoparticles to produce visible light, which activates a photosensitizer attached to the UCNPs to produce singlet oxygen, which destroys the endosomal membrane, promoting the delivery and release of siRNA molecules into cytoplasm for an effective treatment of tumors located deeply in tissue.

### BACKGROUND

RNA interference (RNAi) is a biological mechanism whereby the presence of double-stranded RNA (dsRNA) interferes with the expression of a particular gene that shares a homologous sequence with the dsRNA. In the cytoplasm of mammalian cells, an enzyme known as Dicer initiates RNA silencing by the breakdown of long dsRNA to generate small interfering RNA (siRNA) molecules of 21-25 nucleotides in length. The resulting siRNA are incorporated into an RNA-induced silencing complex (RISC) and unwound into a single-stranded RNA (ssRNA), which is followed by the degradation of sense strand ssRNA. The RISC, containing a guide or antisense strand, seeks out and binds to complementary mRNA sequences. These mRNA sequences are then cleaved by Argonaute, the enzyme within the RISC responsible for mRNA degradation, which leads to mRNA down-modulation.

RNAi-mediated gene silencing has been an important technology in functional gene analysis due to its high specificity, high efficiency and great facility. In addition, it offers one of the most attractive methods for gene therapy for many diseases, including viral infectious diseases and cancerous tumors. Many types of diseases are potential targets for RNAi-based therapy.

However, in siRNA therapy, the most important challenge is the issue of delivery and release of siRNA in the targeted cells. SiRNA is anionic, hydrophilic and unable to enter cells by passive diffusion mechanisms due to repulsion by the negatively charged cell membrane. Moreover, in vivo delivery of naked siRNA to appropriate disease sites remains a considerable hurdle, owing to rapid enzymatic digestion in plasma and renal elimination. In order to interact with the machinery that induces post-transcriptional gene silencing, siRNA molecules need to enter the cytoplasm of the targeted cells, and one of the key steps towards efficient siRNA silencing is the ability of siRNA molecules to escape from the endosomes into the cytosol of the cells, which still remains a challenge.

Cancer is a group of diseases characterized by the uncontrolled growth and spread of abnormal cells. Untreated, cancer can adversely affect a patient's quality of life and can lead to death. The National Cancer Institute reports that more than 18 million cancer cases have been diagnosed since 1990. Cancer is second only to heart disease as a cause of death in the United States of America. The NCI projected that approximately 563,700 Americans would die of cancer in 2005, more than 1,500 people a day. Currently, many therapies have been used for the treatment of cancerous tumors, but there is always some limitation to their practical applications. For example, radiation therapy is currently one of the most common and efficient treatment for many types of cancers. In North America, more than one-half of all cancer patients receive radiation therapy during the course of their treatment. But, because of high-energy radiation used during the treatment, side effects, such as the damage of normal cells and hair loss, often occur. Additionally, secondary electrons from radiation can create highly reactive chemical radicals in the intracellular compartment. Such radicals can break chemical bonds in normal cellular DNA and cause those cells to lose their ability to reproduce.

Since the 1980s, photodynamic therapy (PDT) has been designated as a promising new modality in the treatment of tumors. In a PDT system, a photosensitizer and visible light are necessary to produce active singlet oxygen, which can kill tumor cells. Some research has shown that, in addition to directly killing tumor cells, PDT appears to shrink or destroy tumors in two other ways. The photosensitizer can damage blood vessels in the tumor, thereby preventing the tumor cells from receiving necessary nutrients. In addition, PDT may activate the immune system to attack the tumor cells. However, the PDT techniques that have been developed are limited in clinical practice because the light needed to activate most photosensitizers cannot pass through more than about one-third of an inch of tissue (about one centimeter). For this reason, PDT is usually used to treat tumors on or just under the skin or on the lining of internal organs or cavities. PDT is less effective in treating deeply located tumors because sufficient light cannot get to these tumors.

Recent research demonstrated that singlet oxygen can rupture endosomal membranes to promote the delivery and release of siRNA molecules into the cytosol. Based on this property, a novel photochemical internalization (PCI) technique has been developed for siRNA therapy. (Sabrina Oliveira et al., Biochimica et Biophysica Acta, 2007, 1768, 1211-1217) One of the major advantages of PCI as a delivery tool is its intracellular site-specific action. siRNA delivery is limited to the desired cells, thereby further reducing non-specific effects.

However, the major challenge for this technique is the difficulty of light to deeply penetrate skin and tissues. With respect to both siRNA delivery and the PDT system, production of singlet oxygen from a photosensitizer for cancer treatment is important, and how to effectively activate a photosensitizer to produce singlet oxygen for the treatment of tumors, especially deeply located tumors, has become a focused issue. To our knowledge, most photosensitizers have absorption bands at wavelength shorter than 700 nm. For example, all porphyrin-derived compounds as commercially available photosensitizers, such as photofrin, have a strong absorption band near 400 nm. To treat the tumors, especially deeply located tumors, visible light with wavelength shorter than 700 nm cannot be directly used as a radiation source to activate photosensitizers located near the area of tumors because most tissue chromophores easily absorb visible light, which cannot effectively produce singlet oxygen.

Recently, a novel material named rare earth-doped up-conversion nanoparticles has been developed. This material has special and interesting optical properties. It can produce fluorescent emission in the visible light region upon excitation using infrared (IR) or near infrared (NIR) light as the irradiation source. IR and NIR light can penetrate tissue to a deeper location with much less absorption by the tissue than visible light. On the basis of the optical properties, the deeply located up-conversion nanoparticles (UCNPs) can be effectively excited by IR or NIR light to produce visible light, which can activate the photosensitizer attached to the nanoparticles to release singlet oxygen. For example, the emission spectrum of NaYF₄:Yb-Er nanoparticles has three spectral bands, centered near 525, 542, and 645 nm. They can be assigned to ²H_{11/2}→⁴I_{15/2}, ⁴I_{15/2}, ⁴S_{3/2}→⁴I_{15/2}, and ⁴F_{9/2}→⁴I_{15/2} transitions of Er³⁺ ion, respectively. Based on this knowledge, we have designed a novel system with a combination of siRNA and PDT for treatment of tumors and other diseases.

### SUMMARY OF THE INVENTION

The present invention provides a composition comprising up-conversion nanoparticles (UCNPs) encapsulated with a silica shell and having a photosensitizer incorporated into the silica shell. In one embodiment, the composition further comprises a targeting molecule attached to the silica shell. In another embodiment, the composition comprises a targeting molecule and a small interfering RNA (siRNA) molecule attached to the silica shell.

The invention also provides pharmaceutical compositions comprising the UCNP compositions and a pharmaceutically acceptable carrier.

The compositions of the invention are used to treat disease in humans and other mammals. In one embodiment, the invention provides a method of treating a solid tumor in a mammal by administering a therapeutically effective amount of a composition of the invention to the mammal and activating the composition by applying infrared radiation or near infrared radiation to the site of the solid tumor in the mammal. In another embodiment, the invention provides method of treating an inflammatory disease in a mammal by administering a therapeutically effective amount of a composition of the invention to the mammal and activating the composition by applying infrared radiation or near infrared radiation to the site of the inflammation in the mammal. In a preferred embodiment, the mammal is a human.

The present invention also provides a composition comprising up-conversion nanoparticles (UCNPs) encapsulated with a silica shell and having a targeting molecule attached to the silica shell. The compositions are used for detecting an analyte in a solution or mixture or for measuring the amount or concentration of the analyte in the solution or mixture. The method comprises the steps of: a) contacting a composition, comprising rare earth-doped UCNPs encapsulated with a silica shell and a targeting molecule attached to the silica shell, with the solution or mixture for a sufficient period of time for the composition to bind with the analyte; b) applying IR or NIR light to the solution or mixture; and c) detecting the presence of fluorescent light.

The invention further provides methods for making the rare earth-doped UCNPs. To make the therapeutic composition, a UCNP with a silica shell and a photosensitizer in the shell is synthesized, and then a targeting molecule is attached to the shell. In one embodiment, an siRNA molecule is also attached to the shell. To make the analytic composition, a UCNP with a silica shell is synthesized, and a targeting molecule is attached to the shell. In one embodiment, a magnetic particle is also attached to the shell.

### BRIEF DESCRIPTION OF THE FIGURES

FIG. 1 shows (Left panel) a TEM image of up-conversion NaYF₄:Yb-Er nanparticles, and (Right panel) a photogram of a NaYF₄:Yb-Er nanoparticle solution irradiated with a 980 nm IR laser. Strong up-conversion fluorescence of nanoparticles in hexane can be observed even under indoor light.

FIG. 2 shows (tipper panel) an emission spectrum and (Lower panel) a X-ray diffraction pattern of up-conversion NaYF₄:Yb-Er nanparticles.

FIG. 3 shows (Left panel) a TEM image of silica-coated NaYF₄:Yb-Er up-conversion nanoparticles and (Right panel) a photogram of the nanoparticles in aqueous solution upon excitation with a 40 mW 980 nm laser. Bright emission can be observed even under in-door light.

FIG. 4 shows (A): a fluorescent photogram showing IR activation of up conversion material (UCM) with a silica shell; (B): human breast carcinoma cell line MDA-MB-435 originated xenograft tumor in a nu/nu mouse; (C) IR is able to penetrate into mouse skin to activate UCM that was subcutaneously injected; and (D) IR is able to penetrate into the xenograft tumor to activate UCM.

FIG. 5 shows N[3-(trimethoxysilyl)-propyl]diethylenetriamine) (TSDT) molecular structure (left) and schematic of surface functionalization of nanoparticles with TSDT (right).

FIG. 6 shows the size distribution of surface-modified up-conversion NaYF₄:Yb-Er nanoparticles measured with DLS. The mean diameter is 53.8 nm.

FIG. 7 shows (Upper) conjugation of a monoclonal antibody, protein, or peptide onto the surface of the UCNPs and (Lower) attachment of siRNA molecules onto the surface of UCNPs.

FIG. 8 shows transfection of Cy3-labelled siRNA-UCNP complex with weight ratio of Cy3-labeled siRNA to UCNPs 0.44 in the two kinds of cell lines (upper) HEK293 and (lower) HepG2 measured under (Left panel) bright field and (Right panel) dark field of a fluorescence microscope.

FIG. 9 shows gene silencing efficiencies of siRNA-UCNP complex or surface-modified UCNPs with or without IR excitation in a PC-3 cell line.

FIG. 10 shows the proposed working mechanism of the IR activated siRNA-UCNP drug system after being delivered into the tumor cells.

FIG. 11 shows direct detection of an analyte with the UCNPs. The labels are the UCNPs with a silica shell, but without a photosensitizer. They are linked to the target molecules using classical linking chemistry, based on the functional groups of the target molecules.

FIG. 12 shows sandwich detection of an analyte, using the UCNPs coated with a silica shell as the label.

FIG. 13 shows competitive detection of an analyte, using the UCNPs coated with a silica shell as the label.

FIG. 14 shows a DNA probe assay, using the UCNPs coated with a silica shell as the label.

### DESCRIPTION OF THE INVENTION

The invention provides certain up-conversion nanoparticles, methods for making the particles, and methods of using the particles to treat or diagnose disease or other conditions in a mammal, such as a human patient. As used herein, up-conversion nanoparticles (UCNPs) are particles with a size from a few nanometers to a few hundred nanometers, which can produce high-energy (short-wavelength) emission light (e.g. visible light) upon excitation with low-energy (long-wavelength) light (e.g. infrared light). This differs from traditional luminescent materials, where high-energy light (e.g. ultraviolet light) is needed for excitation to produce low-energy emission light (e.g. visible light).

The particles are rare-earth doped and encapsulated with a silica shell. In one embodiment, they have a photosensitizer incorporated into the shell. In one aspect of this embodiment, the composition has a targeting molecule attached to the shell. In another aspect of this embodiment, the composition also has a small interfering RNA (siRNA) molecule attached to the shell.

The nanoparticles produce fluorescent emission in the visible light region upon excitation with an infrared (IR) or a near infrared (NIR) light irradiation source. IR and NIR light penetrate tissue deeply with less absorption by the tissue. In one aspect, upon excitation with by a 980 nm IR laser, the UCNPs produce fluorescent emission spectra and luminescent images of the UCNPs.

The UCNPs are made from rare earth elements. In one embodiment, the elements are selected from the group consisting of Yttrium (Y), Holmium (Ho), Erbium (Er), Thulium (Tm), and Ytterbium (Yb). In one aspect of this embodiment, the UCNPs have the formula NaYF4: Yb-Ln, wherein Ln is Er, Tm, or Ho. That is, the UCNPs have one of the following compositions: NaYF4:Yb-Tm, NaYF4:Yb-Er, or NaYF4:Yb-Ho. The mole ratio of element Y is from about 60% to about 90%, that of element Yb from about 10% to about 40%, that of element Ln (Er, Tm, or Ho) from about 0.1% to about 30%.

The photosensitizer is any light-sensitive chemical that produces singlet oxygen when excited with light of a specific wavelength. Singlet oxygen is a very aggressive chemical species that reacts very rapidly with nearby biomolecules. In the compositions of the invention, this facilitates the delivery and release of the siRNA or other therapeutic molecule into the cytosol and the killing of tumor and other cells. In one embodiment, the photosensitizer is an organic dye that produces singlet oxygen when excited with light produced from the UCNPs activated by IR or NIR light. Preferably, the photosensitizer has a strong absorption peak that matches the emission peaks of the up-conversion nanoparticles so as to absorb the light energy from the emission of the UCNPs. Certain Porphyrin derivatives that meet these requirements can be determined by persons skilled in the art, given the teachings contained herein. In principle, the stronger the absorption of photosensitizer, the easier and more efficient it is for light energy from UCNPs to be absorbed because absorption of photosensitizer reflects its sensitivity to light with a certain wavelength. Strong emission from the UCNPs is also desirable. Preferably, each photosensitizer molecule is attached tightly on the UCNPs, with a short distance between photosensitizer molecule and the UCNP, so that energy transfers between them occur efficiently. Without being bound by theory, we believe the preferable distance to be about 1 to about 30 nm, which should result in greater than 50% of the energy being transferred from the donor to the acceptor. Merocyanine 540 and Methylene Blue are examples of photosensitizers in the UCNPs of the invention.

The silica shell has been functionalized to permit the attachment of the siRNA and targeting molecules. In one embodiment, a chemical that provides one utilizable functional group for surface functionalization is used. An example is 3-aminopropyltriethoxysilane. In another embodiment, a chemical that provides two or more utilizable functional groups for surface functionalization is used. Examples are N-[3-(trimethoxysilyl)-propyl]ethylenediamine and N-[3-(trimethoxysilyl)-propyl]diethylenetriamine. Utilizable functional groups include amino groups, carboxyl groups, and halogen atoms (e.g., bromine, chlorine, fluorine, or iodine). Preferred functional groups include —NH2, —NH—, —NR2, —COOH, or —X, where X is a halogen and R is any hydrocarbon chain. The functionalization of the silica shell results in it having functional groups, such as —NH2, —NH—, —NR2, —COOH, or —X, where X is a halogen and R is any hydrocarbon chain.

The siRNA molecules bind to a single stranded RNA molecule, which is a messenger RNA (mRNA) that encodes at least part of a peptide or protein whose activity promotes tumorigenesis, angiogenesis, cell proliferation, anti-apoptosis, or inflammation in a human or other mammal, or which is a micro-RNA (miRNA) whose activity promotes tumorigenesis, angiogenesis, cell proliferation, anti-apoptosis, or inflammation in a human or other mammal. For example, the mRNA may encode a protein that is a pro-tumorigenic pathway protein, a pro-angiogenesis pathway protein, a pro-cell proliferation pathway protein, a pro-inflammation pathway protein, or an anti-apoptotic pathway protein. In one embodiment, the molecule is an oligonucleotide with a length of about 19 to about 35 base pairs. In another embodiment, the molecule is an oligonucleotide with a length of about 19 to about 27 base pairs. In still another embodiment, the molecule is an oligonucleotide with a length of about 21 to about 25 base pairs. In all of these embodiments, the molecule may have blunt ends at both ends, or sticky ends at both ends, or a blunt end at one end and a sticky end at the other. In one particular embodiment, it has blunt ends at both ends.

One example is a 25 mer siRNA duplex targeting the human VEGF gene, hVEGF-25c (sense: 5′-CACAACAAAUGUGAAUGCAGACCAA-3′; Antisense:5′-UUGGUCUGCAUUCACAUUUGUUGUG-3′) as the therapeutic component, or a 21 mer VEGF specific inhibitory duplexes, hVEGF-21a (sense: 5′-UCGAGACCCUGGUGGACAUTT-3′; antisense: 5′-AUGUCCACCAGGGUCUCGATT-3′) as the therapeutic component. The siRNA sequences can be used to silence the gene expression of the disease causing protein. For example, the protein can be a VEGF pathway protein, EGFR pathway protein, MGMT pathway protein, RAF pathway protein, MMP pathway protein, mTOR pathway protein, TGFβ pathway protein, or Cox-2 pathway protein. In one embodiment, the protein is one of the following: VEGF, EGFR, PI3K, AKT, AGT, RAF1, RAS, MAPK, ERK, MGMT, MMP-2, MMP-9, PDGF, PDGFR, IGF-1, HGF, mTOR, Cox-2, or TGFβ1. In another embodiment, the protein is VEGF, EGFR, MGMT, MMP-2, MMP-9, or PDGF. In still another embodiment, the protein is RAF1, mTOR, Cox-2, or TGFβ1.

The UCNPs may include more than one kind of siRNA; i.e., the different siRNAs have different cellular targets. In one embodiment, each UCNP has three different siRNAs that bind to three different targets. For example, they can bind to at least one mRNA molecule and at least one miRNA molecule; they can bind to at least two different mRNA molecules; or they can bind to different mRNA molecules that encode different proteins. The proteins can be in the same cellular pathway or in different cellular pathways.

A targeting molecule is any molecule attached to the UCNP that facilitates its delivery in vitro or in vivo to a mammalian cell or to an analyte in a solution or mixture. In one embodiment, the targeting molecule is an antibody, either a polyclonal or a monoclonal antibody. In a preferred embodiment, the antibodies are monoclonal antibodies with isotypes ranging from IgA, IgD, IgE, IgG to IgM, which are able to bind specifically to the targeted cell surface antigens and other markers. A single chain antibody or a fragment of an antibody can also be used as a targeting molecule.

In another embodiment, the targeting molecule is a peptide or protein. In a preferred embodiment, the peptide is PCSVTCGNGIQVRIK, which targets hepatocellular carcinoma cells. It is obtained from the highly conserved carboxyl terminal of the circumsporozoite protein, which coats sporozoites and assists them in accumulating on hepatocytes in vivo. A disulfide-stabilized RGD peptide (such as Gly-Arg-Gly-Asp-Ser-Pro, GRGDSP or H-ACRGDMFGCA-OH or other peptides with the Arg-Gly-Asp core sequence) can be used to target tumors. The RGD peptide is a specific ligand binding to alpha(v)beta3 and alpha(v)beta5 integrins which overexpress on the surface of endothelium in tumor neovasculature. A recently identified FROP peptide (H-EDYELMDLLAYL-OH) can be used due to its unique tumor and tumor vasculature targeting property. An RVG peptide, such as H-YTIWMPENPRPGTPCDIFTNSRGKRASNG-OH, has the properties of passing through the blood brain barrier and targeting brain cells. Other peptides having tissue and cell specific targeting properties can be identified by persons skilled in the art, given the teachings contained herein.

A UCNP of the invention can also include at least one additional nucleic acid, for example a small interfering RNA oligo, a DNA oligonucleotide, a micro RNA (miRNA) oligo, an aptamer, a plasmid, or an mRNA, or a short oligo nucleotide having a therapeutic effect. The nucleic acid can be used as a therapeutic molecule or as a targeting molecule. In one preferred embodiment, the composition of the invention comprises a rare-earth doped up-conversion nanoparticle encapsulated with a silica shell, a photosensitizer incorporated into the silica shell, a targeting molecule attached to the silica shell, and an siRNA molecule attached to the silica shell. The thickness of the silica shell is from about 5 nm to about 20 nm, and the siRNA molecule is an oligonucleotide with a length of 21-25 base pairs. Preferably, the rare-earth doped nanoparticle comprises NaYF4:Yb-Tm, NaYF4:Yb-Er, or NaYF4:Yb-Ho.

In one embodiment, the silica shell of the UCNP also includes at least one small molecule drug attached by electrostatic interaction. Examples of such drugs include cisplatin, carboplatin, oxaliplatin, paclitaxel, docetaxel, and mitomycin.

The invention also includes the UCNPs of the invention combined with a pharmaceutically acceptable carrier. In one embodiment, the carrier comprises at least one of the following: a glucose solution, a polycationic binding agent, a cationic lipid, a cationic micelle, a cationic polypeptide, a hydrophilic polymer grafted polymer, a non-natural cationic polymer, a cationic polyacetal, a hydrophilic polymer grafted polyacetal, a ligand functionalized cationic polymer, a ligand functionalized-hydrophilic polymer grafted polymer, and a ligand functionalized liposome. In another embodiment, the polymers comprise a biodegradable histidine-lysine polymer, a biodegradable polyester, such as poly(lactic acid) (PLA), poly(glycolic acid) (PGA), and poly(lactic-co-glycolic acid) (PLGA), a polyamidoaminc (PAMAM) dendrimer, a cationic lipid (such as DOTAP), or a PEGylated PEI. In still another embodiment, the carrier is a histidine-lysine copolymer that forms a nanoparticle with the siRNA molecule, wherein the diameter of the nanoparticle is about 100 nm to about 500 nm. In a further embodiment, the ligand comprises one or more of an RGD peptide, such as H-ACRGDMFGCA-OH, an RVG peptide, such as H-YTIWMPENPRPGTPCDIFTNSRGKRASNG-OH, or a FROP peptide, such as H-EDYELMDLLAYL-OH.

The UCNP compositions of the invention are prepared in several steps. In one embodiment, the initial composition is made by synthesizing a UCNP with a silica shell and incorporating a photosensitizer into the shell. Alternatively, the formation of a silica shell and the incorporation of a photosensitizer on the UCNP may be completed in the same reaction step. Then one or more chemicals with functional groups are attached to the shell. This permits the subsequent attachment of the targeting and siRNA molecules to the silica shell.

In one embodiment of the invention, the compositions are prepared by the steps of :
a) synthesizing a rare earth-doped upconversion nanoparticle;
b) adding a silica shell to the nanoparticle;
c) incorporating a photosensitizer into the silica shell, if the application is for therapeutics;
d) functionalizing the surface of the shell of the nanoparticle with an amino compound to provide a functional group and a positive charge on the surface;
e) conjugating a targeting molecule to the surface by a functional group; and
f) absorbing an siRNA molecule to the surface of the shell by electrostatic interaction.

In one embodiment, the photosensitizer is incorporated into the silica shell by a reverse micro-emulsion technique. Then, one or more chemicals with functional groups are attached to the shell to provide a positive charge on the shell. Preferably, the chemicals have at least one amino group and may have other functional groups, including other amino groups, carboxyl groups, or a halogen atom. Examples include 3-aminopropyltriethoxysilane, N-[3-(trimethoxysilyl)-propyl]ethylenediamine, and N-[3-(trimethoxysilyl)-propyl]diethylenetriamine. An antibody may be conjugated to the surface by a coupling reaction with EDC/NHS as the coupling agent for the coupling reaction between amino groups and carboxylic acid groups. The siRNA molecule binds to the positively charged shell by electrostatic attraction.

The UCNPs are synthesized by a process wherein nucleation takes place first at room temperature, and then growth proceeds at higher temperature and wherein solvent is refluxed for a period of time from about 0.5 hr. to about 10 hr. in order to produce nanoparticles with a controlled size and a narrow size distribution. In a preferred embodiment, the synthesis uses yttrium salts as a yttrium source, ytterbium salts as a ytterbium source, erbium salts as a erbium source, thulium salts as a thulium source, and holmium salts as a holmium source. The salts are the corresponding metal chlorides, metal nitrates, metal acetates, or metal carbonates. The solvent is a long-chain fatty acid with a carbon number of 8-25, such as oleic acid or stearic acid, or a mixture of the fatty acid and 1-octadecene or trioctylphosphine oxide, or a long-chain fatty amine, such as oleylamine, or a mixture of the fatty amine and 1-octadecence, or water or alcohol, or a mixture of water and alcohol. If water or alcohol or their mixture is used as a solvent, the resulting product is separated from the reaction solution by centrifugation and re-dispersed into an organic solvent, such as a long-chain fatty acid, a long-chain fatty amine, a mixture of 1-octadecene and a long-chain fatty acid, or a mixture of 1-octadecene and a long-chain fatty amine or trioctylphosphine, and then the resulting solution is heated by increasing the reaction temperature to have the solution refluxed for a period of time to complete the growth of the desired nanoparticles. Sodium citrate or ethylenediamine-tetraacetic acid disodium salt (EDTA) can be added to act as a stabilizer by coating the surface of nanoparticles. If a fatty acid or a mixture of 1-octadecene and a fatty acid is used as a solvent during nucleation, sodium hydroxide with a molar concentration of about 5% to about 15% fatty acid can be added to form sodium cations and fatty acid anions, where the latter act as capping agents to stabilize the nanoparticles.

The compositions of the invention are used to treat disease in humans and other mammals. In one embodiment, the invention provides a method of treating a solid tumor in a mammal by administering a therapeutically effective amount of a composition of the invention to the mammal and activating the composition by applying infrared radiation or near infrared radiation to the site of the solid tumor in the mammal. In one aspect of this embodiment, the solid tumor is a non small cell lung carcinoma, a breast carcinoma, a hepatocyte carcinoma, a renal carcinoma, a prostate carcinoma, or a colorectal carcinoma. In another embodiment, the invention provides method of treating an inflammatory disease in a mammal by administering a therapeutically effective amount of a composition of the invention to the mammal and activating the composition by applying infrared radiation or near infrared radiation to the site of the inflammation in the mammal. In one aspect of this embodiment, the inflammatory disease is inflammatory bowel disease, Crohn's disease, or rheumatoid arthritis.

In one embodiment, the mammal is a human, non-human primate, or rodent, such as a mouse, rat, or guinea pig. Rodents are particularly useful for laboratory experiments with the compositions. In a preferred embodiment, the mammal is a human. The compositions are delivered to the subject in pharmaceutically acceptable carriers known to those skilled in the art by techniques known to those skilled in the art. The methods of administration include intravenous injection, intraperitoneal injection, local subcutaneous injection, intra-cerebral injection, intra-articular injection, and intra-muscular injection.

The invention provides several advantages over current therapy. Using UCNPs and siRNA with PDT therapies simultaneously promotes siRNA delivery and PDT for the treatment of the deeply located solid tumors. The small size of the nanoparticles facilitates their delivery into tumor cells, and their large surface area can be modified with biocompatible functional groups. The up-conversion nanomaterials produce visible light upon excitation with IR or NIR light. The IR and NIR light can penetrate tissue deeply with less absorption by the organism's tissue. IR and NIR light are also less harmful to cells and tissues and reduces the risk of inadvertent tissue destruction. Use of cheaper continuous wave-diode IR and NIR lasers for up-conversion can reduce the cost of therapy.

In another embodiment, the compositions of the invention are rare earth-doped UCNPs encapsulated with a silica shell and having a targeting molecule attached to the silica shell. The targeting molecule is an antibody, a peptide, a protein, or a nucleic acid. In one aspect, the targeting molecule is an antibody. In another aspect, it is a DNA oligonucleotide that is complementary to a nucleic acid that is to be detected, thus permitting this construct to be used as a DNA probe.

The compositions are made by synthesizing a rare earth-doped UCNP with a silica shell and attaching a targeting molecule to the silica shell as described herein above.

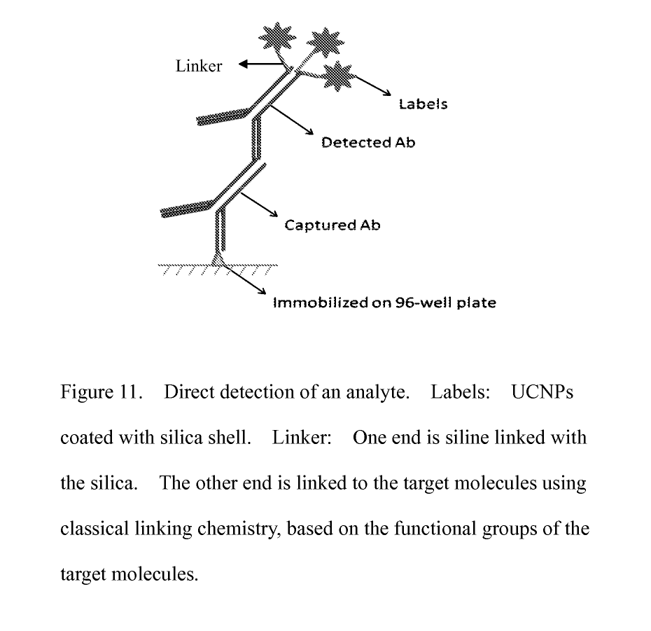

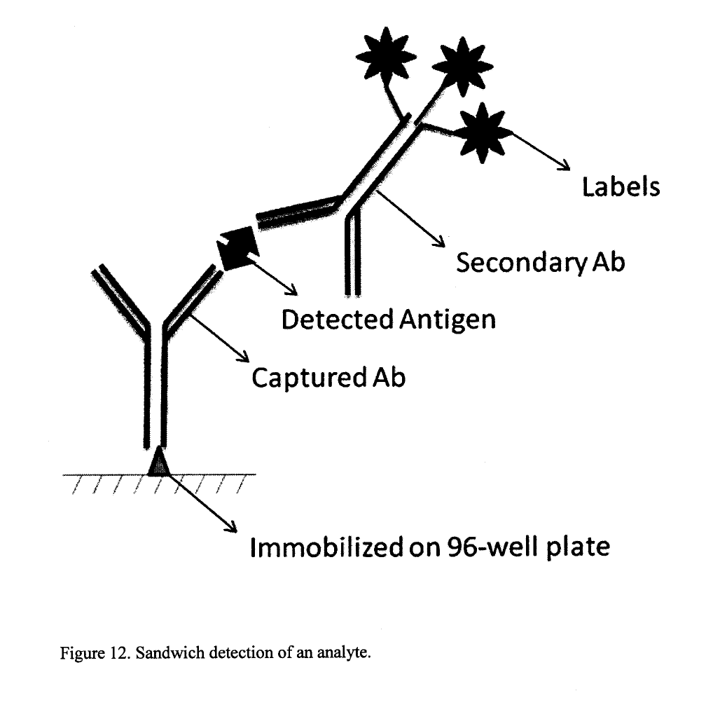

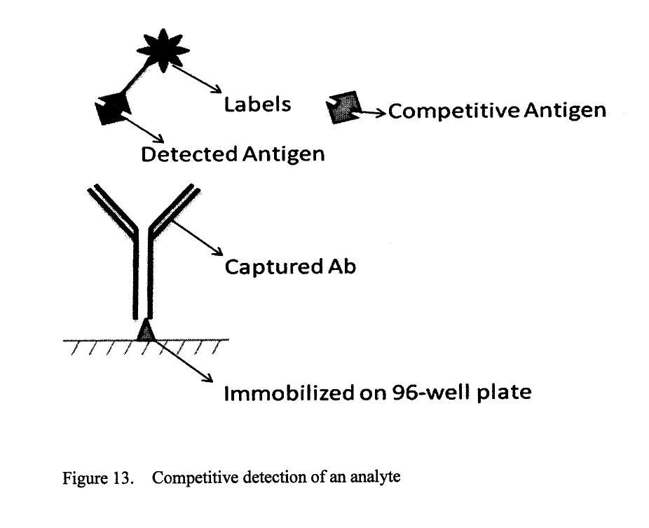

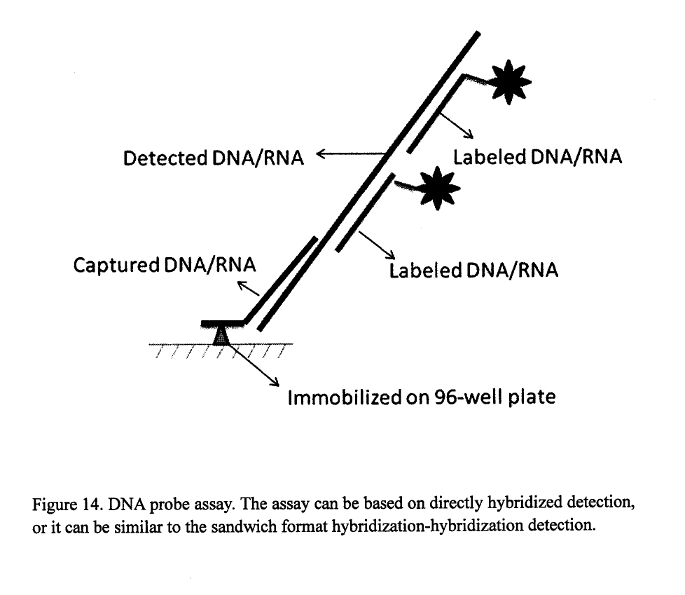

The compositions are used for detecting an analyte in a solution or mixture or for measuring the amount or concentration of the analyte in the solution or mixture. They are especially useful in in vitro diagnostic assays. The assay comprises the steps of: a) contacting a composition, comprising rare earth-doped UCNPs encapsulated with a silica shell and having a targeting molecule attached to the silica shell, with the solution or mixture for a sufficient period of time for the UCNPs to bind with the analyte; b) applying IR or NIR light to the solution or mixture; and c) detecting the presence of fluorescent light. Various assay formats can be used. See FIGS. 11-14.

This embodiment of the invention also includes a kit, comprising the UCNP composition, a container for the composition, and instructions for using the kit.

In one aspect, the composition also includes a magnetic particle attached to the silica shell. This allows the use of magnetism to separate compositions that are bound to an analyte from a mixture or solution in which it is found.

### Experimental Section

### Design

We designed a novel siRNA therapeutic for treatment of cancerous tumors with the assistance of rare earth-doped up-conversion nanoparticles by the following steps:
1. Synthesis of up-conversion nanoparticles. Up-conversion nanoparticles have been shown to have lower cytotoxicity and autofluoresence compared to organic dye and quantum dots (S. Jiang, et al. J. R. Soc. Interface. 2010, 7, 3-18).
2. Attachment of a photosensitizer to the nanoparticle surface by the formation of a porous silica shell, from which the singlet oxygen produced by light-activated photosensitizer can be released;
3. Surface functionalization of the nanoparticles with an amino compound, which can provide positive charge for electrostatic siRNA binding and also for protecting nucleic acids from enzymatic degradation;
4. Conjugation of antibodies to the nanoparticles for effective delivery to target cells; and
5. Absorption of siRNA on the surface of nanoparticles by electrostatic interaction.

### Execution:

1. Synthesis of rare earth-doped upconversion nanoparticles NaYF4: Yb, Ln (Ln=Er, Tm or Ho):

This synthesis is a process in which nucleation takes place first at room temperature and then growth proceeds at higher temperature where the solvent is refluxed for a period of time from about 0.5 hr. to about 10 hr. in order to get nanoparticles with controlled size and narrow size distribution.
1) Yttrium salts act as the yttrium source, ytterbium salts as the ytterbium source, erbium salts as the erbium source, thulium salts as the thulium source, and holmium salts as the holmium source. These salts are the corresponding metal chlorides, corresponding metal nitrates, corresponding metal acetates, or corresponding metal carbonates. The fluoride source can be sodium fluoride or ammonia fluoride.
2) In the total molecular formula, the mole ratio of element Y ranges from 60% to 90%, that of element Yb from 10% to 40%, that of element Ln (Ln=Er, Tm, Ho) from 0.1% to 30%. For example, if Y is 80% and Yb is 18%, then Ln should be 2%.
3) The total concentration of cationic reactants can vary from 0.01 mol/L to 0.1 mol/L. The concentration of anionic reactant(s) can vary from 0.04 mol/L to 10 mol/L.
4) Upon nucleation, the solvent can be long-chain fatty acids with carbon number 8-25, such as oleic acid, stearic acid or a mixture of the fatty acid and 1-octadecene or trioctylphosphine oxide or long-chain fatty amine such oleylamine, or a mixture of the fatty amine and 1-octadecence or water or alcohol or mixture of water and alcohol.
5) During nucleation, if water or alcohol or their mixture is used as the solvent, the resulting product should be separated from the reaction solution by centrifugation and re-dispersed into the organic solvent, such as long-chain fatty acid or long-chain fatty amine or the mixture of 1-octadecene and long-chain fatty acid or the mixture of 1-octadecene and long-chain fatty amine or trioctylphosphine, and then the resulting solution is heated by increasing the reaction temperature to have the solution refluxed for a period of time from 0.5 hr. to 10 hr. to complete the growth of the desired nanoparticles.
6) During nucleation, if the other (organic) solvent mentioned above, except for water, alcohol or their mixture, is used as the reaction solvent, the resulting product can be separated first from the reaction solution by centrifugation and then re-dispersed into the organic solvent such as long-chain fatty acid or long-chain fatty amine or the mixture of 1-octadecene and long-chain fatty acid or the mixture of 1-octadecene and long-chain fatty amine or trioctylphosphinc, and then the resulting solution is heated by increasing the reaction temperature to have solution refluxed for a period of time from 0.5 hr. to 10 hr. to complete the growth of the desired nanoparticles.
7) Alternatively, the resulting product doesn't need to be separated from the reaction solution and directly heat the reaction solution including the resulting product by increasing reaction temperature to have the solution refluxed for a period of time from 0.5 hr. to 10 hr to complete the growth of the desired nanoparticles.
8) During nucleation, if water or alcohol or their mixture is used as a solvent, sodium citrate or ethylenediamine-tetraacetic acid disodium salt (EDTA) is needed to act as a stabilizer to coat the surface of nanoparticles. The amount of sodium citrate or EDTA is 80% to 120% of that of total cationic reactants.
9) During nucleation, if fatty acid or a mixture of 1-octadecene and fatty acid is used as the solvent, sodium hydroxide with mole of 5%-15% fatty acid should be added to form sodium cations and fatty acid anions. The latter are capping agents to stabilize the nanoparticles.
10) Before adding the fluorine source into reaction system at room temperature, the reaction solution should be heated to get solid cationic reactants dissolved completely in the solvent.

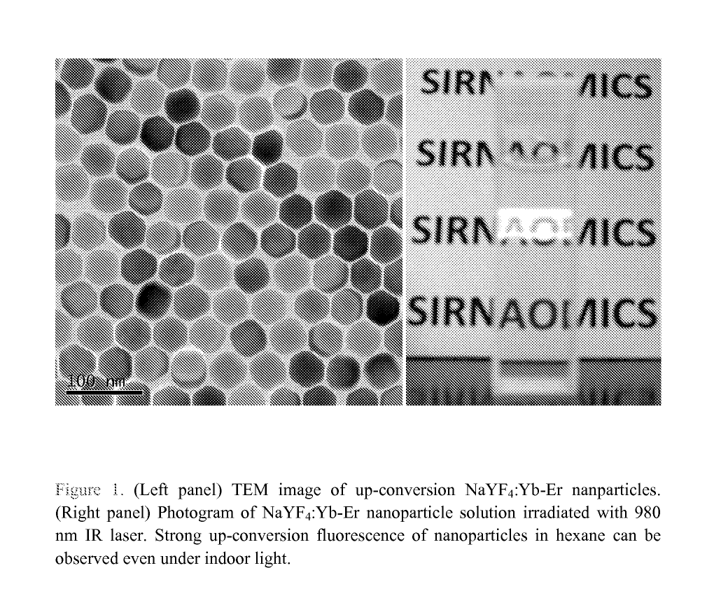

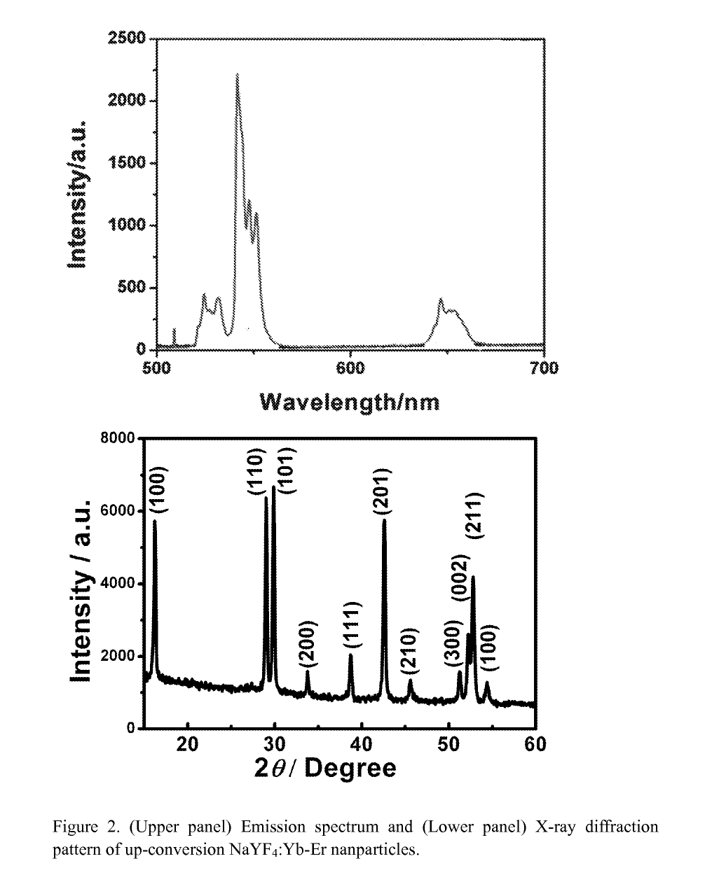

We have synthesized high-quality up-conversion nanoparticles with a controllable size from 10 nm to 200 nm by different approaches. FIG. 1 (left panel) shows a TEM image of ˜42 nm NaYF4:Yb-Er nanoparticles prepared by an organic-phase approach. From FIG. 1 (right panel), it can be seen that the synthesized nanoparticles can be dissolved into a polar solvent to form a transparent solution, and a bright green fluorescent emission can be observed even under indoor light when excited with a 980 nm IR laser. FIG. 2 shows (upper panel) the emission spectrum and (lower panel) wide angle X-ray powder diffraction pattern of up-conversion NaYF₄:Yb-Er nanoparticles. It is noted that there is a strongest peak near 541 nm in the spectrum, and the nanoparticles have a pure hexagonal crystalline structure.

2. Attachment of a photosensitizer to the up-conversion nanoparticles:

A photosensitizer is a light-sensitive chemical which can produce singlet oxygen when excited with the light of specific wavelength. Singlet oxygen is a very aggressive chemical species and will very rapidly react with nearby biomolecules, facilitating the delivery and release of siRNA into cytosol, killing tumor cells. To effectively activate the photosensitizer to produce singlet oxygen, the selected photosensitizer should have a strong absorption peak, matching the emission peak positions of the up-conversion nanoparticles so as to sufficiently absorb the light energy from the emission of up-conversion nanoparticles. In addition, to effectively activate the photosensitizer to produce singlet oxygen, the photosensitizer molecules should be attached tightly on the up-conversion nanoparticles, and there should be a short distance between photosensitizer molecule and nanoparticle so that energy transfer between them can proceed efficiently.

We have designed a method to incorporate a photosensitizer into a porous thin layer of a silica shell on an up-conversion nanoparticle by a reverse microemulsion technique.
1) In this reverse microemulsion system, a nonpolar organic chemical, such as cyclohexane, toluene, or hexane, acts as the organic phase, and water or aqueous solution as the water phase. The surfactant can be Igepal®CO-520, Poly(ethylene glycol) 4-nonylphenyl 3-sulfopropyl ether potassium salt, Synperonic NP-5 or Triton X-100. (Sigma-Aldrich)
2) In this process, the photosensitizer is incorporated into a silica shell formed on the up-conversion nanoparticle surface.
3) The photosensitizer can be hydrophilic or hydrophobic. If it is hydrophilic, aqueous solution is added into the reaction system for its incorporation in the silica shell; if hydrophobic, cyclohexane solution is added to the reaction system for its incorporation in the silica shell.
4) Tetraethyl orthosilicate (TEOS) is used as a silicon source, and ammonia aqueous solution as a catalyst to prompt hydrolysis of TEOS to form the silica shell.
5) The thickness of the silica shell can be controlled by changing the reaction time and the amount of TEOS.

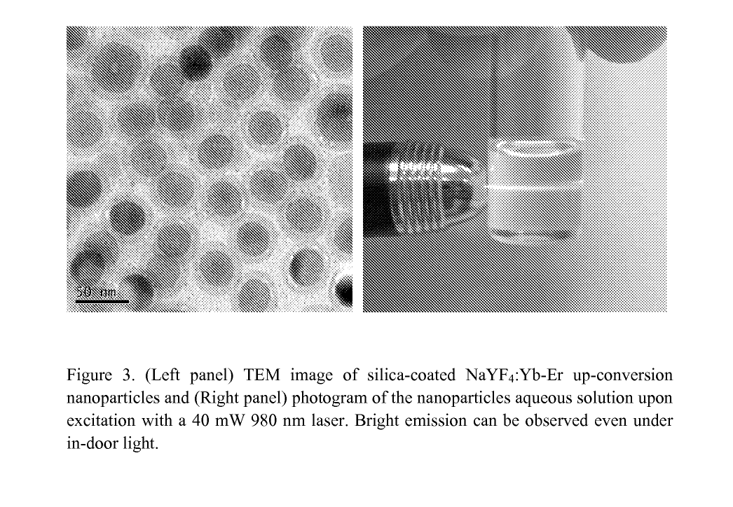

FIG. 3 (left panel) shows a TEM image of silica-coated NaYF₄:Yb-Er up-conversion nanoparticles. Their average size is about 49 nm, wherein the thickness of silica shell on the particle surface is about 7 nm. The silica-coated nanopartcles become hydrophilic and can be dispersed in water very well. FIG. 3 (right panel) shows the photogram of silica-coated NaYF₄:Yb-Er up-conversion nanoparticles aqueous solution, the bright emission light can be observed even under indoor light upon excitation with a 980 nm laser.

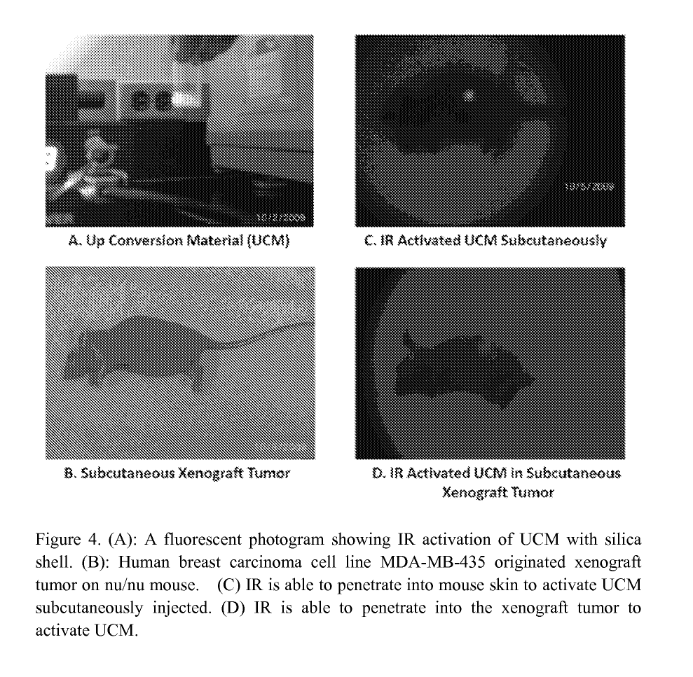

We conducted in vivo validation of IR activation of the silica-coated, rare earth-doped up-conversion material (UCM) using a mouse xenograft tumor model. The UCM was injected into the flank tumor mass with 300 cubic mm in volume (FIG. 4). The IR laser at 980 nm was given to the tumor site, resulting in fluorescence emission which can be observed with either the naked eye or a camera. FIG. 4(A) shows a fluorescent photogram showing IR activation of UCM with silica shell. FIG. 4(B) shows human breast carcinoma cell line MDA-MB-435 originated xenograft tumor on nu/nu mouse. FIG. 4(C) shows IR is able to penetrate into mouse skin to activate UCM subcutaneously injected. FIG. 4(D) shows IR is able to penetrate into the xenograft tumor to activate UCM.

3. Surface functionalization of nanoparticles:

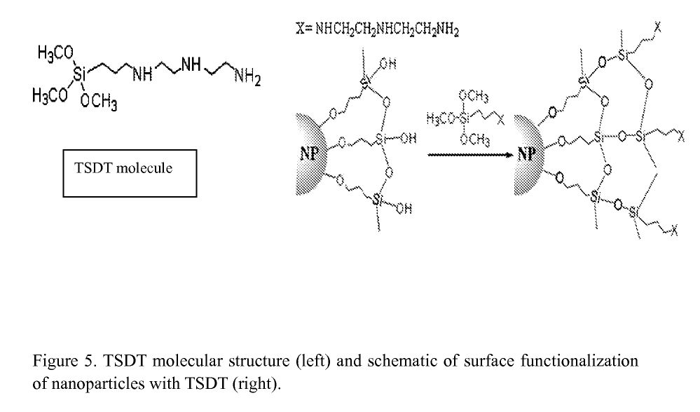

After the formation of the silica shell, the up-conversion nanoparticles convert from hydrophobic to hydrophilic, and the particle surface has rich —Si—O—H groups, which can further react with chemicals with alkyloxysilyl groups to form —O—Si—O— bonds. To functionalize the surface of the nanoparticles, some chemicals with both alkyloxysilyl groups and other functional groups, such as —NH₂, —NH—, —NR₂, —COOH, or —X, where X is a halogen and R is any hydrocarbon chain, can be used to modify the surface.
1) In this process, the selected chemicals for surface functionalization can be chemicals with one functional group, such as —H₂, —NH—, —NR₂, —COOH, or —X (for example: 3-aminopropyltriethoxysilane), or chemicals with two or more functional groups concurrently (for example: N-[3-(trimethoxysilyl)-propyl]ethylenediamine and N-[3-(trimethoxysilyl)-propyl]diethylenetriamine). FIG. 5 shows the TSDT molecular structure (left) and a schematic of surface functionalization of the nanoparticles with TSDT (right).
2) In this process, two or more chemicals with different groups (for example, one chemical has a carboxylic group, while the other has an amino group, which can be a primary amino group, secondary amino group, or tertiary amino group) can be used concurrently for surface functionalization.
3) In this process, the chemicals with at least one amino group and other functional group, such as the other amino group(s), or carboxylic group or halogen atom, can be chosen for surface modification so that an antibody or a peptide, and the siRNA molecules can be conjugated to the UCNPs in later steps.
4) The selected chemical for surface functionalization can be added to the above reaction system for the formation of silica shell at the late stage of the reaction, when the the silica shell with a certain thickness has formed. Generally, the preferred thickness for the shell is from about 5 nm to about 20 nm. The thickness can be controlled by reaction conditions, such as the concentration of the reactions, reaction time, and the temperature. Higher concentration, longer reaction time, and higher temperature lead to a thicker shell.
5) The selected chemical for surface functionalization can be added to a new reaction system, where the purified silica-coating nanoparticles are re-dispersed in water, with a little (20-200 μl) aqueous ammonia solution added as catalyst.

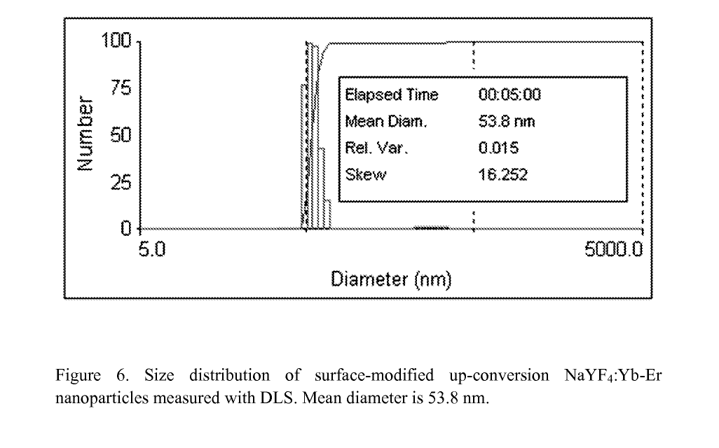

FIG. 6 shows the size distribution of surface-modified up-conversion NaYF₄:Yb-Er nanoparticles, which was measured using a dynamic light scattering (DLS) particle size analyzer. The mean size is about 54 nm, close to the size of the silica-coated nanoparticles without functional molecules modified on the particle surface. This suggests that the surface-modified nanoparticles can be dispersed very well with fewer aggregations in solvent (water here).

4. Conjugation of antibody molecules to the nanoparticles:

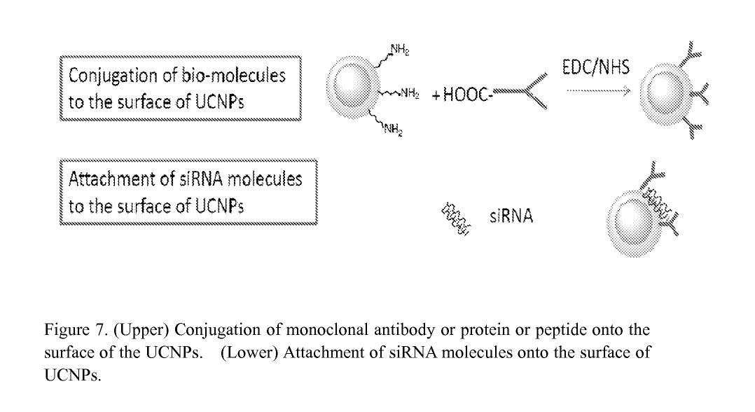

To efficiently deliver the functionalized nanoparticles to the target cells, antibody, peptide, or other targeting molecules are conjugated to the surface of nanoparticles by a coupling reaction. For example, EDC/NHS are effective coupling agents for the coupling reaction between amino groups and carboxylic acid groups. (See FIG. 7.)
1) After antibody molecules are conjugated to nanoparticles, the molecules for surface functionalization should have remaining amino groups unreacted with antibodies or peptides for siRNA molecules to be attached to the nanoparticles.
2) The remaining amino groups can be primary amino groups, secondary amino groups, or tertiary amino groups.
   5. Absorption of siRNA on the surface of nanoparticles:

The remaining amino groups on the nanoparticle surfaces are positively charged sites, which can absorb negatively charged siRNA molecules. If the selected molecule for surface functionalization in step 3 is one with both a primary amino group and a secondary amino group except for alkyloxysilyl groups, for example N-[3-(trimethoxysilyl)-propyl]diethylenetriamine), during the coupling reaction, the carboxylic acid groups in antibody molecules preferentially react with the active primary groups, while secondary amino groups remain unreacted. The latter can absorb siRNA molecules by electrostatic interactions.

The following example illustrates certain aspects of the invention and should not be construed as limiting the scope thereof.

### EXAMPLE

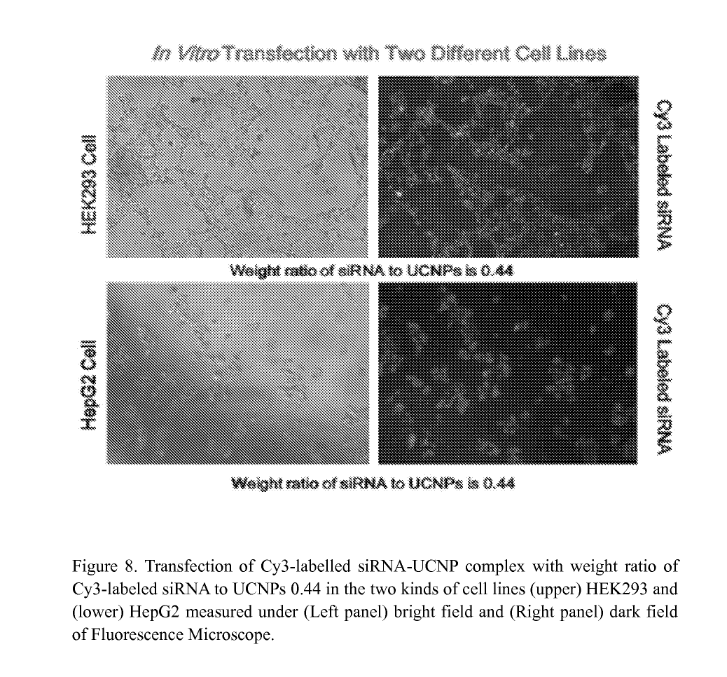

Cellular transfection was performed using the silica-coated, rare earth-doped UCNPs with photosensitizers in the silica shell and modified with functional molecules, such as TSDT on the particle surface. A certain amount of the surface modified UCNP solutions were mixed with Cy3 labeled siRNA with the desired ratios to prepare siRNA-UCNP complexes. In vitro transfection of the novel delivery system was studied. As shown in FIG. 8, the two cell lines, HEK293 and HepG2, were used for studying the transfection efficacy under a fluorescence microscope, and the obtained results suggest an effective transfection of the cells.

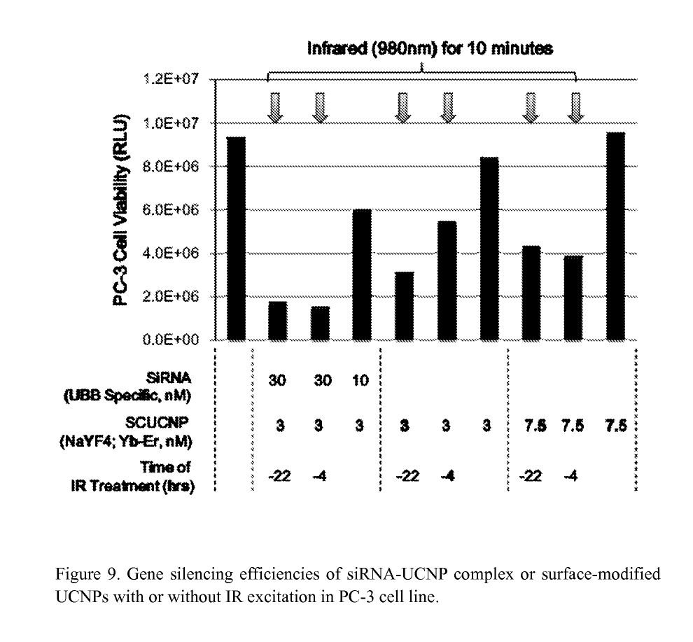

Then, this delivery system was used to explore gene silencing efficiencies. We chose PC-3 cell line for some control experiments under different conditions for comparison of gene silencing efficiencies, and the results are shown in FIG. 9. It is noted that, when siRNA-UCNPs were used for the transfection of PC-3 cell line with IR irradiation on the cells, the gene silencing efficiency is the best, which is indicated by the lowest cell viability in FIG. 9. It is likely that both singlet oxygen released from the IR-activated drug system and siRNA molecules escaping from ruptured endosomes into the cytosol of the cells work together in killing cancerous cells. If only the surface modified UCNPs without siRNA were used for the transfection of PC-3 cell line, and IR irradiation was imposed on the cells, the gene silencing efficiency is also apparent, which is likely due to the singlet oxygen produced from the light-activated photosensitizer molecules; while without IR light to irradiate the cells, the surface-modified UCNPs cannot play a role in killing cancerous cells. This experiment also proves a good transfection efficacy of siRNA-UCNP complexes in the cells.

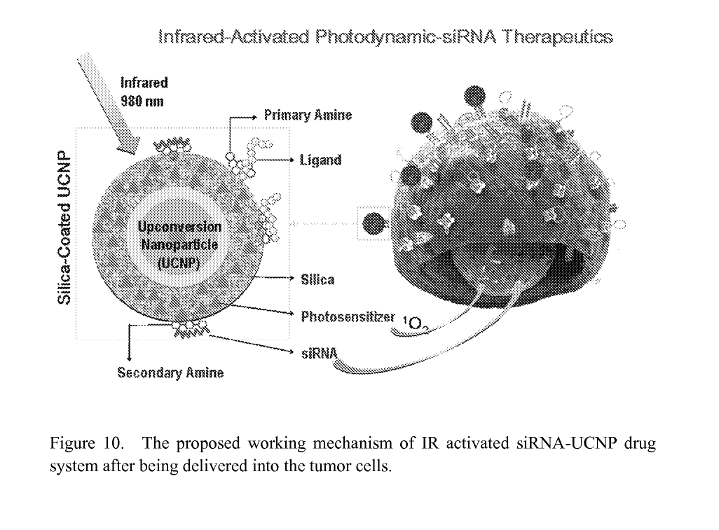

FIG. 10 shows the proposed working mechanism of the IR activated siRNA-UCNP drug system after being delivered into the tumor cells. After IR activates UCNPs, the singlet oxygen released from the particle will help to break the cell membrane and promote endosomal escape of siRNA content following receptor-mediated endocytosis.

### NIR Spectroscopy Application

The basis for near-infrared NIR spectroscopy and imaging in breast cancer has been that tumor alterations of tissue vascularization/angiogenesis and oxygen consumption can be measured through hemoglobin concentration and oxygenation state, respectively. Several investigators have developed diffuse optical imaging DOI and diffuse optical spectroscopy DOS instruments at discrete wavelengths in the 650- to 980-nm range to detect and characterize breast tumors due to the absorption of both oxy- and deoxyhemoglobin. NIR tissue absorption spectra are typically fitted with hemoglobin extinction spectra obtained in vitro to quantify tissue hemoglobin concentrations.

Fiber optic sensors based on near infrared (NIR) diffuse optical spectroscopy have the potential to improve the sampling yield of image-guided core needle biopsy. In the NIR spectral region between 650 -1000 nm, the number of light scattering events in tissue is approximately two orders of magnitude greater than the number of absorption events. This allows light to penetrate up to several centimeters into breast tissues before being absorbed by the tissue or collected by a detector. The NIR absorption and scattering properties of tissue can be quantitatively described using a model of light propagation based on the diffusion approximation to the radiative transport equation. The diffusion equation can be used to calculate the absorption and scattering coefficients of tissue from NIR spectroscopic measurements of diffusely reflected light, from which tissue composition can be derived. Endogenous absorbers in breast tissue include oxygenated hemoglobin (HbO2), deoxygenated hemoglobin (Hb), water and lipids. Endogenous scattering is associated with microscopic variations in the size, shape and refractive indices of both intracellular and extracellular components. Tissue vascularity, hemoglobin saturation and water content have all been identified as diagnostic markers of breast cancer using a variety of different techniques including immunohistochemistry, needle oxygen electrodes and magnetic resonance spectroscopy. Breast cancers are more vascularized, have hypoxic regions and an elevated water content compared to normal breast tissues. Thus, NIR diffuse optical spectroscopy offers a rapid and quantitative assessment of tissue physiological and structural properties for characterizing breast tissue composition and for the diagnosis of breast cancer in vivo. NIR diffuse optical spectroscopy has been widely used for intact breast tumor characterization, monitoring of tumors in the intact breast in response to neoadjuvant chemotherapy, quantifying the effects of menopausal status on breast tissue properties, and breast tissue perfusion studies. NIR diffuse optical spectroscopy can be implemented for breast cancer diagnosis and therapeutics.

### REFERENCES

1. Bumcrot D, Manoharan M, Koteliansky V, Sah D W Y. RNAi therapeutics: a potential new class of pharmaceutical drugs. Nature Chemical Biology, 2006, 2, 711-9.
2. Endoh T, Ohtsuki T. Cellular siRNA delivery using cell-penetrating peptides modified for endosomal escape. Adv. Drug Deliv. Rev. 2009, 61, 704-9.
3. Oh Y K, Park T G. siRNA delivery system for cancer treatment. Adv. Drug Deliv. Rev. 2009, 61, 850-62.
4. Yezhelyev M V, Qi L, O'Regan R M, Nie S, Gao X. Proton-sponge coated quantum dots for siRNA delivery and intracellular imaging. J. Am. Chem. Soc. 2008, 130, 9006-12.
5. Jiang S, Gnanasammandhan M K, Zhang Y. Optical imaging-guided cancer therapy with fluorescent nanoparticles. J. R. Soc. Interface, 2009.
6. Oliveira S, Fretz M M, Hogset A, Storm G, Schiffelers R M. Photochemical internalization enhances silencing of epidermal growth factor receptor through improved endosomal escape of siRNA. Biochimica et Biophysica Acta 2007,1768, 1211-7.
7. He X, Wang K, Tan W, Liu B, Lin X, He C, Li D, Huang S, Li J. Bioconjugated nanoparticles for DNA protection from cleavage. J. Am. Chem. Soc. 2003, 125, 7168-9.
8. Bharali D J, Klejbor I, Stachowiak E K, Dutta P, Roy I, Kaur N, Bergey E J, Prasad P N, Stachowiak M K. Organically modified silica nanoparticles: a nonviral vector for in vivo gene delivery and expression in the brain. Proc. Natl Acad. Sci. USA 2005, 102, 11539-44.
9. Zhang P, Steelant W, Kumar M, Scholfield M. Versatile photosensitizers for photodynamic therapy at infrared excitation. J. Am. Chem. Soc. 2007, 129, 4526-7.
10. Li Q, Zhang Y, Jiang S. Multicolor core/shell-structure upconversion fluorescent nanoparticles. Adv. Mater. 2008, 20, 4765-9.

All publications, including issued patents and published patent applications, and all database entries, identified by url addresses or accession numbers, are incorporated herein by reference in their entirety.

Although this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A composition comprising rare earth-doped up-conversion nanoparticles (UCNPs) encapsulated with a silica shell wherein a photosensitizer is incorporated into the silica shell.

2. The composition of claim 1 further comprising a targeting molecule attached to the silica shell.

3. The composition of claim 2 wherein the rare earth element is selected from the group consisting of Yttrium (Y), Holmium (Ho), Erbium (Er), Thulium (Tm), and Ytterbium (Yb).

4. The composition of claim 3 wherein the UCNPs have the formula NaYF4: Yb-Ln, wherein Ln is Er, Tm, or Ho.

5. (canceled)

6. The composition of claim 2 wherein the UCNPs produce fluorescent emission in the visible light region upon excitation with an infrared (IR) or a near infrared (NIR) light irradiation source.

7. The composition of claim 2 wherein the photosensitizer comprises a light-sensitive chemical that produces singlet oxygen when excited with light of a specific wavelength.

8.-10. (canceled)

11. The composition of claim 2 wherein the silica shell comprises functional groups with —O—Si—O bonds.

12. The composition of claim 2 wherein the silica shell is functionalized with a chemical with one functional group.

13. The composition of claim 2 wherein the silica shell is functionalized with a chemical with two or more functional groups.

14.-17. (canceled)

18. The composition of claim 2 wherein the targeting molecule comprises an antibody.

19. (canceled)

20. The composition of claim 2 wherein the targeting molecule comprises a protein or peptide.

21.-22. (canceled)

23. The composition of claim 2 wherein the targeting molecule is a nucleic acid.

24. The composition of claim 1 further comprising a small interfering RNA (siRNA) molecule attached to the silica shell.

25.-29. (canceled)

30. The composition of claim 24 further comprising an additional nucleic acid.

31. (canceled)

32. The composition of claim 24 wherein the silica shell further comprises at least one small molecule drug attached by electrostatic interaction.

33. (canceled)

34. A composition comprising a rare earth-doped up-conversion nanoparticle encapsulated with a silica shell, a photosensitizer incorporated into the silica shell, a targeting molecule attached to the silica shell, and a small interfering RNA (siRNA) molecule attached to the silica shell.

35. The composition of claim 34 wherein the thickness of the silica shell is from about 5 nm to about 20 nm.

36. The composition of claim 34 wherein the rare-earth doped nanoparticle comprises NaYF4:Yb-Tm, NaYF4:Yb-Er, or NaYF4:Yb-Ho.

37. (canceled)

38. A pharmaceutical composition comprising the composition of claim 24 and a pharmaceutically acceptable carrier.

39. (canceled)

40. A method of treating a solid tumor in a mammal comprising the steps of administering a therapeutically effective amount of the composition of claim 24 to the mammal and activating the composition by applying infrared radiation or near infrared radiation to the site of the solid tumor in the mammal.

41.-42. (canceled)

43. A method of treating an inflammatory disease in a mammal comprising the step of administering a therapeutically effective amount of the composition of claim 24 to the mammal and activating the composition by applying infrared radiation or near infrared radiation to the site of the inflammation in the mammal.

44.-49. (canceled)

50. A method for making a UCNP composition comprising the steps of: a) synthesizing a UCNP with a silica shell; and b) incorporating a photosensitizer into the shell.

51.-64. (canceled)

65. A composition comprising rare earth-doped UCNPs encapsulated with a silica shell and a targeting molecule attached to the silica shell.

66. The composition of claim 65 further comprising a magnetic particle attached to the silica shell.

67. The composition of claim 65 wherein the targeting molecule is an antibody, a peptide, a protein, or a nucleic acid.

68.-69. (canceled)

70. A method of making the composition of claim 65 comprising the steps of: a) synthesizing a UCNP with a silica shell; and b) attaching a targeting molecule to the silica shell.

71. The method of claim 70 comprising the additional step of attaching a magnetic particle to the silica shell.

72. A method for detecting an analyte in a solution or mixture or for measuring the amount or concentration of the analyte in the solution or mixture comprising the steps of : a) contacting the composition of claim 65 with the solution for a sufficient period of time for the composition to bind with the analyte; b) applying IR or NIR light to the solution; and c) detecting the presence of fluorescent light.

73. The method of claim 72 further comprising the step of determining the amount or concentration of the analyte from the intensity of the light.

74. A kit comprising the composition of claim 65, a container for the composition, and instructions for using the kit.


SEQUENCE LISTING (9 sequences)

SEQ ID NO: 1 - RNA, 25 nt, Artificial Sequence
  Description of Artificial Sequence Synthetic oligonucleotide
cacaacaaaugugaaugcagaccaa

SEQ ID NO: 2 - RNA, 25 nt, Artificial Sequence
  Description of Artificial Sequence Synthetic oligonucleotide
uuggucugcauucacauuuguugug

SEQ ID NO: 3 - DNA, 21 bp, Artificial Sequence
  Description of Artificial Sequence Synthetic oligonucleotide
ucgagacccugguggacautt

SEQ ID NO: 4 - DNA, 21 bp, Artificial Sequence
  Description of Artificial Sequence Synthetic oligonucleotide
auguccaccagggucucgatt

SEQ ID NO: 5 - protein, 15 aa, Artificial Sequence
  Description of Artificial Sequence Synthetic peptide
PCSVTCGNGIQVRIK

SEQ ID NO: 6 - protein, 6 aa, Artificial Sequence
  Description of Artificial Sequence Synthetic peptide
GRGDSP

SEQ ID NO: 7 - protein, 10 aa, Artificial Sequence
  Description of Artificial Sequence Synthetic peptide
ACRGDMFGCA

SEQ ID NO: 8 - protein, 12 aa, Artificial Sequence
  Description of Artificial Sequence Synthetic peptide
EDYELMDLLAYL

SEQ ID NO: 9 - protein, 29 aa, Artificial Sequence
  Description of Artificial Sequence Synthetic peptide
YTIWMPENPRPGTPCDIFTNSRGKRASNG